(19) United States
(12) Patent Application Publication
(10) Pub. No.: US 2011/0247657 A1
(43) Pub. Date: Oct. 13, 2011

(54) Rinse Aid, Comprising Fatty Acid Alkanolamide Polyalkylene Glycol Ethers

(75) Inventors: Frederic Bauer, Dusseldorf (DE); Sabine Both, Neuss (DE); Teresa Alexandre, Dusseldorf (DE); Melanie Nagel, Oberhausen (DE); Snsgar Behler, Bottrop (DE); Frank Clasen, Hiden (DE)

(73) Assignee: Cognis IP Management GmbH, Dusseldorf (DE)

(21) Appl. No.: 13/140,265

(22) PCT Filed: Dec. 11, 2009
(86) PCT No.: PCT/EP2009/008867

§ 371 Date: Jun. 16, 2011

(30) Foreign Application Priority Data
Dec. 20, 2008 (EP) EP08022221

(51) Int. Cl. A47L 15/42; C07C 233/05; C07C 233/18; C11D 3/60

(52) U.S. Cl. 134/18; 510/514; 554/35; 554/61; 554/64

(57) ABSTRACT

Provided for are rinse aids that exhibit improved drying properties during automatic dish washing and thus allow cleaning methods at lower temperatures than customer reference agents. The rinse aids comprise a) at least one surfactant according to the general formula (I), where R¹ is a saturated or unsaturated, branched or linear alkyl or alkenyl radical having 6 to 22 carbon atoms, R² is hydrogen or an alkyl group having 1 to 6 carbon atoms or a group R³, and R³ is a group A-(O—CH₂CHR⁴—)ₘ—OR⁵ and R⁴ and R⁵ independently from each other are a hydrogen atom or an alkyl group having 1 to 4 carbon atoms and A is a (CH₂)ₙ— group or a hydroxyl alkyl group having 2 to 6 carbon atoms and n is an integer from 1 to 6 and m is an integer or a fractional number from 1 to 10 and furthermore b) at least one hydrotrope, c) at least one organic hydroxycarboxylic acid and d) water.

The present application relates to rinse aids for automatic dishwashing which must comprise certain fatty acid alkanolamide polyalkylene glycol ethers, and to the use of these amides for improving the drying performance of dishwashing detergents.

Nowadays, higher demands are placed on machine-washed dishes than on manually washed dishes. For example, even dishes completely cleaned of food residues are not assessed as perfect if, after machine dishwashing, they still have whitish spots based on water hardness or other mineral salts which, due to a lack of wetting agent, originate from dried water drops. In order to obtain brilliantly clear and spot-free dishes, rinse aids are therefore used. The addition of liquid or solid rinse aid ensures that the water runs off from the wear as completely as possible, meaning that the wide variety of surfaces are residue-free and shiny at the end of the wash programme. Standard commercial rinse aids are mixtures of nonionic surfactants, solubility promoters, organic acids and solvents, water and optionally preservatives and fragrances. The aim of the surfactants in these compositions consists in influencing the interfacial tension of the water in such a way that it can run off from the ware in the thinnest possible, coherent film so that no water drops, streaks or films are left behind during the subsequent drying process (so-called wetting effect). A distinction is generally made here between two types of deposits: firstly, the so-called “spotting” is investigated, which arises as a result of drying water drops, and secondly “filming” is evaluated, i.e. layers which are formed by the drying of thin water films. For the evaluation, test persons are currently used who visually evaluate the parameters of “spotting” and “filming” on cleaned objects, e.g. plates, glasses, knives etc. Modern dishwashing detergents therefore comprise rinse aids in order to improve the run-off of water from the surfaces of the ware. However, there are rinse aids which do not dry equally as well on all substrates, such as e.g. plastic. In order to get round this effect, complex rinse aids are formulated which have e.g. silicone compounds and/or fluorinated compounds, as are described in U.S. Pat. No. 5,880,089 or US 2005/0143280 A1. However, these compounds are biodegradable with difficulty, if at all, and some are even hazardous to the environment.

More recently, moreover, combination products have increasingly been used; these combine the different functions, such as cleaning, clear rinsing, water softening and optionally metal protection, in particular silver protection, or a glass protection function in one, preferably solid, supply form. Such compositions are referred to as multifunctional compositions. Thus, for example so-called three-in-one products which combine cleaner, rinse aid and water softener in the form of a solid compact (“tab”) are found on the market. As a result of the increased use of such multifunctional compositions, however, the drying performance has become worse compared to using a classic rinse aid. Drying performance is to be understood here as meaning to what extent the cleaned ware still has water, preferably water drops, on the surface after passing through the dishwashing process. The water remaining on the surface then has to either be removed mechanically (e.g. by wiping dry) or the ware has to be left to dry in the air. The user thus has to wait until the water has evaporated. In this connection, however, residues (e.g. lime and/or surfactant residues or other residues which were dissolved or dispersed in the water) remain on the surface and lead to unaesthetic spots or streaks. This is true to a particular extent on shiny or transparent surface, such as e.g. glass or metal. For improved drying in a dishwasher, an extension or intensification of the drying phase would also in principle be conceivable, e.g. as a result of increasing the temperature in the drying step. However, not all ware withstands higher temperatures, and plastic articles in particular may be sensitive. An extension of the drying phase is in most cases perceived as a disadvantage by users.

Moreover, such measures lead to an increased energy consumption of the machine. However, it is desired to avoid the latter since energy consumption is becoming an ever more important selling criterion for dishwashing detergents. For some time, consumers have already been paying more and more attention to the energy consumption of domestic appliances. There are even already standards, inter alia for energy consumption and drying performance, thus e.g. the standard ÖVN/ÖNORM EN 50242 +A1+A2+A3, edition 2003-11-01, valid for Austria, or the contextually similar standard for Germany, DIN EN 50242. Here in particular the edition 2005-06 “Electric dishwashers for household use—measurement methods for use properties (IEC 60436:2004, modified); German version EN 50242:2004).

There is therefore an increased need for technical solutions for making dishwashing processes more energy-saving. Such solutions may also be the use of dishwashing detergents, or additives in such compositions, which shorten the high energy-expenditure drying step, or make it possible to dry at a lower temperature.

Ways are therefore sought to improve the drying performance of cleaners for hard surfaces, in particular of dishwashing detergents for automatic dishwashing, where, here, in particular those solutions are sought in which the temperature in the clear-rinse cycle can be reduced.

It has been found that through the co-use of certain fatty acid alkanolamide polyalkylene glycol ethers in rinse aids or dishwashing detergents, the aforementioned problem can be solved.

In a first embodiment, the application relates to rinse aids comprising at least a) one surfactant according to the general formula (I)

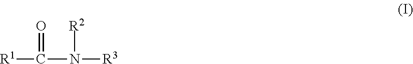

in which R¹ is a saturated or unsaturated, branched or linear alkyl or alkenyl radical having 6 to 22 carbon atoms, R² is hydrogen or an alkyl radical having 1 to 6 carbon atoms or a radical R³, and R³ is a radical A-(O—CH₂—CHR⁴—)ₘ—OR⁵ and R⁴ and R⁵, independently of one another, are a hydrogen atom or an alkyl radical having to 4 carbon atoms, and A is a group (CH₂)ₘ— or a hydroxyalkyl group having 2 to 6 carbon atoms, and n is an integer from 1 to 6, and m is integers or fractional numbers from 1 to 10, b) is a hydrotope, c) is an organic hydroxycarboxylic acid and d) is water.

The compounds a) of the formula (I) are known per se and can be described as fatty acid alkanolamide polyalkylene glycol ethers. DE 102 59 405 A1 describes the preparation of such light-colored amides by alkoxylation of fatty acid alkanolamides. This specification describes a process for the preparation of light-colored fatty acid alkanolamide polyalkylene glycol ethers according to the above general formula (I), in which an addition of alkylene oxides onto fatty acid alkanolamides takes place in the presence of alkaline catalysts, which is characterized in that the alkoxylation is carried out in the presence of reducing agents and the reaction products obtained in this way are then subjected to a steam treatment under alkaline conditions.

WO 00/08125 A1 discloses rinse aids must comprise surfactants, hydrotopes and a polymer. As possible surfactants, purely ethoxylated carboxamides having at most 3 parts of ethylene oxide are specifically disclosed, without the specification disclosing the advantages of selecting this surfactant class for the energy efficiency of clear-rinse processes.

The selection of the fatty acid alkanolamides used, which are condensation products of technical-grade fatty acids with mono- or dialkanolamines, is not critical per se. Typically, the starting materials used are those fatty acid alkanolamides which conform to the formula R′CO—NR″R′″, in which R′CO is a linear or branched, saturated or unsaturated acyl radical having 6 to 22 carbon atoms and 0 or 1 to 3 double bonds, R′ is a hydroxyalkyl group having 2 to 4 carbon atoms and R″ and R′″ are hydrogen or R′. Typical examples are the condensation products of caproic acid, caprylic acid, capric acid, lauric acid, myristic acid, palmitic acid, stearic acid, isostearic acid, oleic acid, linoleic acid, linolenic acid, petroselic acid, elaeostearic acid, 12-hydroxystearic acid, ricinoleic acid, gadoleic acid, arachidonic acid, behenic acid, erucic acid, and also technical-grade mixtures thereof, in particular coconut fatty acid, palm kernel fatty acid, palm fatty acid and tallow fatty acid with monoethanolamine, diethanolamine, monopropanolamine and dipropanolamine, and mixtures thereof. Preference is given to using condensation products of coconut or tallow fatty acids with monoethanolamine. These condensation products are then reacted with alkoxides, e.g. as per the teaching in DE 102 59 405 A1.

Suitable alkoxides are ethylene oxide (EO), propylene oxide (PO) and butylene oxide (BO) or mixtures thereof. The compounds of the formula (I) can either be reacted only with one alkoxide, or different alkoxides may be present alongside one another in the molecules. In the case of such mixed alkoxides, preference is given in particular to those which have ethylene oxide and propylene oxide alongside one another.

In mixed alkoxylates, the different alkoxylates may be distributed blockwise or irregularly (“random distribution”). The preparation takes place by alkoxylating condensation products of alkanolamides with fatty acids in a manner known per se, particular preference being given to products which have been prepared according to the process of the aforementioned DE 102 59 405 A1. DE A 1 692 015 discloses carboxamides having 8-20 carbon atoms, and also addition products thereof onto less than 5 parts of ethylene oxide, with an addition product of a C12/C14 carboxamide with 3.5 parts of ethylene oxide being specifically described here. The latter can be excluded from the protection of the present application.

Preferred amides a) are characterized structurally in that, in the formula (I), R¹ is a linear, saturated alkyl radical having 8 to 18 carbon atoms and R⁴ is hydrogen, n is 2 and m is an integer or fractional number between 1 and 6. Moreover, preference is given to those amides a) in which, in the formula (I), the radical R³ has the meaning (CH₂)ₙ—(OC₂H₄)ₓ(OC₃H₆)_{y}—OH, where n is the numbers specified above and x is preferably numbers from 1 to 6, preferably up to 4, and y is integers or fractional numbers from 1 to 6, preferably up to 4, or has the value zero, with the proviso that the sum of x and y is at most 10 and preferably at most 8, in particular at most 5. As a preferred lower limit of the sum of x and y (or only x, if y=zero), an even or fractional number from 3 to 8, preferably from 4 to 7, may be selected. The distribution of alkoxylates here may take place blockwise or randomly. The depiction of the group (OC₂H₄)ₓ and (OC₃H₆)_{y} in the above formulae therefore also include all other conceivable sequences of the alkoxylates.

Particular preference is given here to those compounds in which the index y has a value from 1 to 6, but is not zero, i.e. compounds which are ethoxylated and propoxylated. Preference is then given to those ethoxylated and propoxylated compounds of the general formula (I) in which 1 to 4 parts of propylene oxide and 2 to 3 parts of ethylene oxide are present. Preferred compounds contain e.g. 3 parts of ethylene oxide and 1, 2 or 4 parts of propylene oxide, or 2 parts of ethylene oxide and 1 part of propylene oxide. Further preferred compounds of the general formula (I) are the products of examples 1 and 2 disclosed in DE 102 59 405 A1, paragraphs [0012] to [0014] of the specification.

Typical examples of compounds according to the formula (I) are alkoxylates of the condensation products of caproic acid, caprylic acid, capric acid, lauric acid, myristic acid, palmitic acid, stearic acid, isostearic acid, oleic acid, linoleic acid, linolenic acid, petroselic acid, elaeostearic acid, 12-hydroxystearic acid, ricinoleic acid, gadoleic acid, arachidonic acid, behenic acid, erucic acid, and technical-grade mixtures thereof, in particular coconut fatty acid, palm kernel fatty acid, palm fatty acid and tallow fatty acid with monoethanolamine, diethanolamine, monopropanolamine and dipropanolamine, and mixtures thereof. Preference is given to using condensation products of coconut or tallow fatty acids with monoethanolamine in alkoxylated form.

The compounds according to a) may be mono- or dialkylated amides. In the case of monoalkylamides, R² in the formula (I) is a hydrogen atom, in the case of the dialkylamides, R² and R³ are in each case independently of one another radicals of the structure —(CH₂)ₙ—(O—CH₂—CHR⁴—)ₘ—OR⁵, it being possible for the precise structure, thus e.g. the number and distribution of the alkoxide groups in the radicals R² and R³, to be different.

Preferred amides are those which have ethylene oxide and propylene oxide groups alongside one another in the molecule, the fraction of the ethylene oxide groups preferably being higher than the fraction of the propylene oxide groups. Particularly preferred compounds of the formula (I) contain 3 to 4 parts of ethylene oxide and 2 to 3 parts of propylene oxide. Preference is also generally given to those compounds of the formula (I) in which the radical R⁵ is a hydrogen atom.

Compounds which contain no propylene oxide units may be less advantageous. Thus, one preferred embodiment of the invention relates to those rinse aid compositions in which the compounds of the formula (I) obligatorily contain ethylene oxide and propylene oxide groups. It may also be preferred to use those compounds of the general formula (I) in which the index x has a higher value than the index, or that y has a higher value than x, or that x and y are the same.

DE A 1692015 describes compositions which comprise no hydrotopes, but adducts of carboxamides having 8 to 20 carbon atoms with less than 5 mol of ethylene oxide. The compositions of the present invention may preferably be free from these adducts.

As a further component, the rinse-aid compositions comprise a hydrotrope, preferably selected from the group of cumenesulfonates, xylenesulfonates, propylene glycols and here preferably 1,2-monopropylene glycol, alkyl(oligo)glycosides, ethoxylated alkyl (oligo)glycosides, alkylated alkyl (oligo)glycosides, alkyl phosphates and/or fatty alcohol alkoxylates, preference being given to cumene- and xylenesulfonates and the alkyl (oligo)glucosides. Hydrotropes are agents which act as solubility promoter for a second, more sparingly soluble substance.

Cumene- and xylenesulfonates are to be regarded as particularly preferred component b), here in particular the sodium salts of cumene- or xylenesulfonate. Particular preference is given to cumenesulfonate, sodium salt.

Alkyl and alkenyl oligoglycosides (which are supplied by the applicant under the parent mark APG®) are known nonionic surfactants which to the formula R⁶O-[G]ₚ, in which R¹ is an alkyl and/or alkenyl radical having 4 to carbon atoms, G is a sugar radical having 5 or 6 carbon atoms and p is numbers from 1 to 10. They can be obtained by the relevant methods of preparative organic chemistry. The alkyl and/or alkenyl oligoglycosides can be derived from aldoses or ketoses having 5 or 6 carbon atoms, preferably from glucose. The preferred alkyl and/or alkenyl oligoglycosides are therefore alkyl and/or alkenyl oligoglucosides. The index number p in the general formula gives the degree of oligomerization (DP), i.e. the distribution of mono- and oligoglycosides, and is a number between 1 and 10. Whereas p in a given compound must always be an integer and here in particular can assume the values p=1 to 6, the value p for a specific alkyl oligoglycoside is an analytically ascertained calculated parameter, which in most cases is a fractional number. Preference is given to using alkyl and/or alkenyl oligoglycosides with an average degree of oligomerization p of from 1.1 to 3.0. From the point of view of application, preference is given to those alkyl and/or alkenyl oligoglycosides whose degree of oligomerization is less than 1.7 and is in particular between 1.2 and 1.4. The alkyl or alkenyl radical R⁶ can be derived from primary alcohols having 4 to 11, preferably 8 to 10, carbon atoms. Typical examples are butanol, caproic alcohol, caprylic alcohol, capric alcohol and undecyl alcohol, and also technical-grade mixtures thereof, as are obtained for example during the hydrogenation of technical-grade fatty acid methyl esters or in the course of the hydrogenation of aldehydes from the Roelen oxo synthesis. Preference is given to alkyl (oligo)glucosides based on the chain length C₈-C_{10 (DP=}1 to 3), which are produced as forerunning during the distillative separation of technical-grade C₈-C₁₈-coconut fatty alcohol and may be contaminated with a fraction of less than 6% by weight of C₁₋₂-alcohol, and also alkyl (oligo)glucosides based on technical-grade C_{9/11}-oxo alcohols (DP=1 to 3). The alkyl or alkenyl radical R⁶ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Preference is given to alkyl (oligo)glucosides based on hydrogenated C_{12/14}-coconut alcohol with a DP of 1 to 3. A particularly preferred alkyl (oligo)glycoside is the product from Cognis which is supplied under the name Glucopon® 215 UP. It is an alkyl (oligo)glucoside, where R⁶ is linear alkyl radicals having 6 to 8 carbon atoms, the degree of DP is between 1.1 and 1.2.

Besides the above-described APG® which preferably have linear alkyl radicals, it is also possible to use those alkyl (oligo)glycosides which contain branched alkyl radicals. Such compounds are disclosed e.g. in US H171, or described in WO 94/21655 A1. Alkyl (oligo)glycosides based on branched alcohols, preferably on the Guerbet alcohol type, are advantageous, where alkyl (oligo)glycosides based on 2-ethylhexanol and 2-propylheptanol may be particularly preferred.

Also suitable are alkoxylated alkyl (oligo)glycosides which correspond to the formula (II)

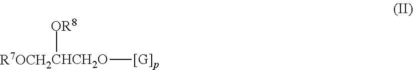

in which R⁷ is an optionally hydroxyl-functionalized alkyl, alkenyl or acyl radical having 6 to 22 carbon atoms, R⁸ is hydrogen or R⁷, G is a sugar radical having 5 to 12 carbon atoms and p is numbers from 1 to 10. The preparation of such alkoxylates is described in DE 197 28 900 A1.

Further suitable hydrotropes b) are methylated alkyl (oligo)glycosides, as are disclosed e.g. in the earlier, previously unpublished European patent application No. 08007291.1. These are alkyl and/or alkenyl ether mixtures of alkyl and/or alkenyl polyglycosides of the formula (III)

(Gₘ-R⁹)R¹⁰ₙ (III)

in which G is a sugar radical having 5 to 6 carbon atoms, R⁹ is a C₆ to C₂₂ alkyl and/or alkenyl radical in acetal bond, R¹⁰ is a C1 to C4 alkyl and/or alkenyl group in ether bond, m is an average value from 1.2 to 1.8, and n is a number from 1.4 to 2.6, where preferably at least 50% by weight of the alkyl and/or alkenyl ethers contain a radical R⁹ with a carbon chain greater than or equal to 12. The compounds of the formula (III) are prepared by reacting alkyl (oligo)glycosides with alkylating reagents such as alkenyl halides and/or alkyl or alkenyl tosylates and/or dialkyl or dialkenyl sulfates.

Further suitable hydrotropes b) are also the alkyl esters of phosphoric acid, preferably the mono- or dialkyl esters, where the alkyl radicals preferably have 6 to 12 and in particular 8 to 12 carbon atoms.

Another suitable group of hydrotropes b) are the alkoxylated fatty alcohols. As a consequence of the preparation, fatty alcohol ethoxylates are referred to as fatty alcohol or oxo alcohol alkoxylates and preferably conform to the formula (IV),

R¹¹O(CH2CHR¹²O)_{q}H (IV)

in which R¹¹ is a linear or branched alkyl and/or alkenyl radical having 6 to 22 carbon atoms, R¹² is hydrogen or an alkyl radical having 1 to 3 carbon atoms and q is numbers from 1 to 50. The fatty alcohol alkoxylates according to the formula (IV) can preferably contain ethylene oxide groups, propylene oxide groups or both alkoxides, where these may be present blockwise (first one block of ethylene oxide, then one block of propylene oxide, or vice versa; or the molecule contains several blocks of different alkoxylates in succession, where a block is to be understood as meaning at least two identical alkoxide group) or in random distribution. The preparation takes place in the manner known to the person skilled in the art by reacting fatty alcohols with the alkoxides in the presence of acidic or basic catalysts. Within the context of the present technical teaching, fatty alcohol alkoxides which are ethoxylated and propoxylated are preferred.

As third component c), the rinse aids according to the invention comprise an acid and here usually organic hydroxycarboxylic acids which are preferably selected from the group mandelic acid, lactic acid, hydroxysuccinic acid, citric acid and tartaric acid, with particular preference being given to citric acid.

In addition, the rinse aids according to the invention also comprise water, preferably in amounts, based on the total weight of the compositions, of from 10 to 90% by weight, preferably from 40 to 75% by weight and in particular from 50 to 65% by weight. Preference is given here to using deionized water. The amide component a) is preferably present in amounts of from 1 to 80% by weight, preferably 10 to 50% by weight and in particular from 20 to 45% by weight, based on the weight of the total composition. The hydrotropes b) are preferably present in amounts of from 1 to 30% by weight, preferably 1 to 20% by weight and in particular from 1 to 10% by weight, based on the weight of the total composition. The acid component c) is preferably present in amounts of from 1 to 10% by weight, preferably from 1 to 5% by weight, based on the weight of the total rinse aid.

Particularly preferred rinse aids comprise the compounds of the formula (I) together with citric acid and either cumenesulfonates or alkyl (oligo)glucosides.

The compositions of the present invention can also comprise polymers, although it may be preferred for the compositions to be free from polymers, in particular free from polymers which comprise poly(meth)acrylic acid monomers.

The compositions are prepared by mixing the different components, optionally with the input of energy by stirring and/or heating of the mixtures. Preferably, after introducing the water as initial charge, the remaining components are added in any order with stirring and then the mixture is further stirred until clear.

Besides the essential components a) to c) and water, further ingredients that are customary per se may also be present in the rinse aids according to the invention. These are preferably selected from the group of alcohols and here preferably the short-chain alcohols ethanol, propanol, isopropanol, butanol, isobutanol. Moreover, the compositions can also comprise further surfactants, such as e.g. fatty alcohol alkoxylates or alkylpyrrolidones, polymers, e.g. polycarboxylates or preservatives, e.g. isothiazolinones. The co-use of phosphates, here in particular of alkyl phosphates, is also possible.

The compositions can, however, also comprise perfumes, dyes, UV protection, e.g. benzophenones, pH regulators and further customary additives, e.g. those for preventing glass or metal corrosion and for protecting metals against tarnishing. The pH of the compositions can be in the range from pH 3 to 10, preferably from pH 3.5 to 8 and particularly preferably from 4 to 7 or 4 to 6. It is generally the case that acidic compositions (pH<7) may be preferred.

However, preference is given to those aqueous compositions which comprise only the components a) to c). Furthermore, it may be advantageous to dispense with the presence of hydrocarbons and in particular terpenes. In one preferred embodiment of the invention, the compositions are therefore free from terpenes and/or hydrocarbons.

The present application further provides the use of compounds of the general formula (I) according to claim for producing aqueous rinse aids, or for producing dishwashing detergents. Particular preference is moreover given to the use of compounds of the general formula (I) according to the above description for improving the drying performance of rinse aids for dishwashing, or of dishwashing detergents.

The fatty acid alkanolamide polyalkylene glycol ethers according to the formula (I) can be incorporated into customary rinse aids and in particular dishwashing detergents on their own or in combination with the components a) to c). The dishwashing detergents may be solid, or in pressed form, e.g. as a tablet, or be liquid or gel-like. The compounds of the formula (I) are present in ready-formulated dishwashing detergents preferably in amounts of from 1 to 45% by weight, in particular in amounts of from 4 to 30% by weight and particularly preferably in amounts of from 15 to 30% by weight.

Such ready-formulated, thus standard commercial dishwashing detergents comprise e.g. builder materials, surfactants, bleaches, bleach activators, enzymes, enzyme stabilizers, corrosion inhibitors, film inhibitors, complexing agents, inorganic salts, graying inhibitors, foam inhibitors, silicone oils, soil release compounds, color transfer inhibitors, salts of polyphosphonic acids, optical brighteners, fluorescent agents, disinfectants, fragrances, dyes, antistatics, ironing auxiliaries, phobicization and impregnation agents, swelling and nonslip agents, UV absorbers, pH regulators, or a mixture thereof.

Typical formulations of such compositions for machine dishwashing comprise pentasodium triphosphate (s. sodium phosphates) in amounts of 0, or 1 to 50% by weight. Sodium citrate in amounts of from 30 to 45% by weight, sodium polycarboxylate in amounts of from 5 to 8% by weight, sodium metasilicate in amounts of from 10 to 60% by weight, 0 sodium hydrogen carbonate in amounts of from 10 to 25% by weight, sodium disilicate in amounts of from 10 to 30% by weight, sodium perborate in amounts of from 5 to 10% by weight, bleach activators in amounts of from 2 to 4% by weight, enzymes, preferably protease and amylase, in amounts of from 1 to 6% by weight, low-foam nonionic surfactants in amounts of from 1 to 20% by weight, silver/glass protection and fragrances in amounts up to 5% by weight. Suitable surfactants are anionic, nonionic, cationic and/or amphoteric surfactants, where nonionic surfactants and here low-foam nonionic surfactants may be particularly preferred. The latter are preferably selected from the class of fatty alcohol alkoxylate, preferably the fatty alcohol ethoxylates, where end-group-capped derivatives may also be suitable.

As a consequence of the preparation, fatty alcohol ethoxylates are referred to as fatty alcohol or oxo alcohol ethoxylates and preferably conform to the formula RO(CH₂CH₂O)ₙH, in which R is a linear or branched alkyl and/or alkenyl radical having 6 to 22 carbon atoms and n is numbers from 1 to 50. Typical examples are the adducts of, on average, 1 to 50, preferably 5 to 40 and in particular 10 to 25, mol of caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and also technical-grade mixtures thereof which are produced e.g. during the high-pressure hydrogenation of technical-grade methyl esters based on fats and oils or aldehydes from the Roelen oxo synthesis, and also as monomer fraction during the dimerization of unsaturated fatty alcohols. Preference is given to adducts of from 10 to 40 mol of ethylene oxide onto technical-grade fatty alcohols having 12 to 18 carbon atoms, such as, for example, coconut, palm, palm kernel or tallow fatty alcohol.

The fatty acid alkanolamide polyalkylene glycol ethers of the formula (I) show, alone or preferably in combination with hydrotropes b), in rinse aids or in dishwashing detergents during automatic dishwashing, an improved drying behavior on porcelain, glass, metal and plastic (e.g. in the case of Tupperware, SAN (=styreneacrylonitrile), melamine, polypropylene, polyethylene). The improved drying performance on glass and metal substrates is particularly well pronounced. The improved drying behavior is moreover more pronounced when using low-temperature programmes or saving or eco programmes with a reduced maximum temperature in the clear-rinse cycle and/or in the drying step. In one preferred embodiment, the compounds of the formula (I) are used rinse aids or dishwashing detergents as additives which serve to improve the drying performance and/or the cleaning performance. The amides of the general formula (I) can be used here either in aqueous compositions, preferably in aqueous rinse aids, or else in solid compositions. In general, the amides of the formula (I) are suitable for producing rinse aids or dishwashing detergents.

The present application therefore further provides a method for automatic dishwashing, where the ware passes through at least one cleaning cycle using a detergent and water, at least one clear-rinse cycle using a rinse aid and water, and a final drying cycle, where the temperature in the drying cycle does not exceed 50° C., where, in step i) and/or ii), a composition comprising a compound of the formula (I) as per the above description is used. By using the amides according to the formula (I), the drying temperatures can be reduced from the 70 to 60° C. otherwise customary in such processes to 55° C. or even lower, e.g. down to 45 or 50° C.

The use of the amides of the formula (I) in dishwashing detergents and/or in rinse aids thus permits a notable saving of energy compared with current dishwashing processes and compositions since, on account of the improved drying behavior, a shorter drying time or preferably a lower drying temperature, preferably less than 60° C. and in particular from 40 to 59° C., very particularly preferably from 45 to 55° C., can be used. Typical values for an energy saving, which can be achieved for in each case one run of the entire washing process, are preferably 0.05 to 0.2 kWh, compared with the same process but which a reference rinse aid without the amide a) essential to the invention. The reference rinse aid selected is the rinse aid according to the description in ÖVE/ÖNORM EN 50242 (edition: 2003-11-01) Annex B.2 rinse aid [formula III]. The washing process according to the above description is suitable for meeting the requirements placed on drying performance and energy consumption according to the German standard DIN EN 50242.

### EXAMPLES

To test the application properties of the fatty acid alkanolamide polyalkylene glycol ethers according to the invention, the following formulations 1 to 8 were prepared (all percentages refer to % by weight, based on the active substance content) by introducing the ingredients one after the other into water with stirring. EO stands for ethylene oxide units, PO for propylene oxide units.

### Formulation 1:

40% amide according to formula (I) with 3EO/2PO based on coconut fatty acids 4% alkyl polyglucoside C₈₋₁₀, (Glucopon® 215 UP, Cognis)
5% citric acid
51% deionized water

### Formulation 2:

40% amide according to formula (I) with 3EO/2PO based on coconut fatty acids
4% alkylphosphate C8-10 (Tresolit® AP-36, Cognis)
5% citric acid
51% deionized water

### Formulation 3:

40% amide according to formula (I) with 3EO/2PO based on coconut fatty acids
4% sodium cumenesulfonate
5% citric acid
51% deionized water

### Formulation 4:

40% amide according to formula (I) with 3EO/4PO based on coconut fatty acids
4% APG methyl ether (Eumulgin® GTS, Cognis)
5% citric acid
51% deionized water

### Formulation 5:

40% amide according to formula (I) with 3EO/4PO based on coconut fatty acids
4% alkyl polyglucoside C₈₋₁₀, (Glucopon® 215 UP, Cognis)
5% citric acid
51% deionized water

### Formulation 6:

33% amide according to formula (I) with 2EO/1PO based on coconut fatty acids
11% alkyl polyglucoside C₈₋₁₀, (Glucopon® 215 UP, Cognis)
5% citric acid
51% deionized water

### Formulation 7:

28% amide according to formula (I) with 2EO/1PO based on coconut fatty acids
16% alkyl polyglucoside C₈₋₁₀, (Glucopon® 215 UP, Cognis)
5% citric acid
51% deionized water

### Formulation 8:

26.7% amide according to formula (I) with 3EO/2PO based on coconut fatty acids
13.3% C12-14-fatty alcohol with 5EO/4PO (Dehypon®LS 54, Cognis)
4% sodium cumenesulfonate
5% citric acid
51% deionized water

### Reference Rinse Aid:

EN 50242 B.2 rinse aid [formula III]

Using the formulations 1 to 8, parts of kitchen- or tableware made of glass, porcelain, metal (=cutlery), Tupperware and styreneacrylonitrile plastic were then washed, in each case under identical conditions, in modified AEG Electrolux (Favorit 60870 model) dishwashers on the 50° C. programme without fan, and the ware was then assessed visually for residues/water drops (drying performance according to Cognis).

Using the formulation 1, parts of kitchen- or tableware made of glass, porcelain, metal (=cutlery), Tupperware and styreneacrylonitrile plastic were washed, likewise under identical conditions, in two standard commercial Miele G 696-2 SC Plus dishwashers on the programmes Economy and Universal 55, and the ware was then assessed visually for residues/water drops (drying performance according to Cognis).

Furthermore, using the formulation 1, parts of kitchen- or tableware made of glass, porcelain, metal (=cutlery) and styreneacrylonitrile plastic were analyzed, under identical conditions, in 3 different standard commercial dishwashers:

a) AEG Favorit 60870, 50° C., hot rinse without fan;
b) Miele G 696-2 SC Plus, “Economy” programme and
c) Bosch SMS65T25EU “active water”, on the programme eco 50° C. The machine of type c) operates with hot air dried by zeolites in the drying step in order to achieve a better drying result on the part of the machine.

The washed ware was then assessed visually for residues/water drops. When assessing the drying performance, each part of the load was assessed individually and graded as follows:

| | |
|---|---|
| dry (part is completely free from moisture) | 0 |
| 1 drop/run trace | 1 |
| 2 drops/run trace | 2 |
| 3 drops/run trace | 3 |
| 4 drops/run trace | 4 |
| 5 drops/run trace | 5 |
| 6 drops/run trace and more | 6 |

### Cups/Bowls/Glasses:

| | |
|---|---|
| Water in glass; cup or bowl cavity | 6 |
| Only “damp” | 3 |
| Dry | 0 |

The results for the three machines can be found in Tables 1a to 1c below. These show the ascertained values for 3 ml of a composition according to the invention, the values for the reference composition, and also a relative assessment where the following apply: values better than the standard: +, same as the standard: 0, worse than the standard:

TABLE 1a
(AEG Favorit 60870):

| | | | | | Average | |
|---|---|---|---|---|---|---|
| | | | | | value | Average |
| | | | | | of all | value |
| | Porce- | | | Cut- | substrate | of all |
| | lain | Plastic | Glass | lery | groups | substrates |
| 3 ml of | 4.9 | 4.4 | 3.7 | 1.7 | 3.7 | 3.4 |
| formulation | | | | | | |
| 1 | | | | | | |
| Compared to | 5.8 | 5.5 | 5.6 | 3.0 | 5.0 | 4.6 |
| 3 ml of | | | | | | |
| EN 50242 B.2 | | | | | | |
| rinse aid | | | | | | |
| [formula | | | | | | |
| III] | | | | | | |
| Compared to | + | ++ | ++ | ++ | ++ | ++ |
| 3 ml of | | | | | | |
| EN 50242 B.2 | | | | | | |
| rinse aid | | | | | | |
| [formula | | | | | | |
| III] | | | | | | |

TABLE 1b
(Miele G 696-2 SC Plus):

| | | | | | Average | |
|---|---|---|---|---|---|---|
| | | | | | value | Average |
| | | | | | of all | value |
| | Porce- | | | Cut- | substrate | of all |
| | lain | Plastic | Glass | lery | groups | substrates |
| 3 ml of | 0.2 | 0.2 | 0.0 | 0.1 | 0.1 | 0.1 |
| formulation | | | | | | |
| 1 | | | | | | |
| Compared to | 0.4 | 0.4 | 0.6 | 0.3 | 0.4 | 0.4 |
| 3 ml of | | | | | | |
| EN 50242 B.2 | | | | | | |
| rinse aid | | | | | | |
| [formula | | | | | | |
| III] | | | | | | |
| Relative | + | + | ++ | + | + | + |
| compared to | | | | | | |
| 3 ml of | | | | | | |
| EN 50242 B.2 | | | | | | |
| rinse aid | | | | | | |
| [formula | | | | | | |
| III] | | | | | | |

TABLE 1c
(Bosch SMS65T25EU “Active Water”):

| | | | | | Average | |
|---|---|---|---|---|---|---|
| | | | | | value | Average |
| | | | | | of all | value |
| | Porce- | | | Cut- | substrate | of all |
| | lain | Plastic | Glass | lery | groups | substrates |
| 3 ml of | 0.0 | 0.1 | 0.0 | 0.0 | 0.0 | 0.0 |
| formulation | | | | | | |
| 1 | | | | | | |
| Compared to | 0.2 | 0.4 | 0.0 | 0.0 | 0.1 | 0.1 |
| 3 ml of | | | | | | |
| EN 50242 B.2 | | | | | | |
| rinse aid | | | | | | |
| [formula | | | | | | |
| III] | | | | | | |
| Relative | + | + | 0 | 0 | + | + |
| compared to | | | | | | |
| 3 ml of | | | | | | |
| EN 50242 B.2 | | | | | | |
| rinse aid | | | | | | |
| [formula | | | | | | |
| III] | | | | | | |

Furthermore, using the formulation 1, special substrate plates made of glass, ceramic, metal, PP=polypropylene, SAN=styreneacrylonitrile plastic were washed, under the same conditions, in two standard commercial Miele G 681 SC Plus dishwashers on the Economy programme, and the substrates were analyzed for residues by means of digital imaging, as described in EP 1 635 167 A1 by the applicant (clear-rinse performance: spotting on PP=polypropylene, stainless steel, glass, ceramic, SAN=styreneacrylonitrile, and also filming on glass and SAN=styreneacrylonitrile). The results are shown in Tables 2a and 2b:

TABLE 2a
(filming)

| | Filming on glass | | | Filming on SAN | | |
|---|---|---|---|---|---|---|
| | | | Consider- | | | Consider- |
| | Slight | Average | able | Slight | Average | able |
| 3 ml of | 98.27 | 99.85 | 99.92 | 65.10 | 91.82 | 98.93 |
| formulation 1 | | | | | | |
| Compared to | 99.23 | 99.91 | 99.93 | 53.00 | 84.30 | 97.10 |
| 3 ml of | | | | | | |
| EN 50242 B.2 | | | | | | |
| rinse aid | | | | | | |
| [formula III] | | | | | | |
| Relative | 0 | 0 | 0 | ++ | ++ | + |
| compared to | | | | | | |
| 3 ml of | | | | | | |
| EN 50242 B.2 | | | | | | |
| rinse aid | | | | | | |
| [formula III] | | | | | | |

TABLE 2B
(spotting)

| | Spotting | | | | |
|---|---|---|---|---|---|
| | Glass | Ceramic | Metal | PP | SAN |
| 3 ml of | 98.49 | 99.05 | 99.03 | 99.37 | 92.14 |
| formulation 1 | | | | | |
| Compared to | 99.27 | 99.25 | 98.84 | 99.35 | 96.68 |
| 3 ml of | | | | | |
| EN 50242 B.2 | | | | | |
| rinse aid | | | | | |
| [formula III] | | | | | |
| Relative | 0 | 0 | 0 | 0 | — |
| compared to | | | | | |
| 3 ml of | | | | | |
| EN 50242 B.2 | | | | | |
| rinse aid | | | | | |
| [formula III] | | | | | |

The investigations show that by using the amides according to the invention, good drying performances can be achieved on all relevant substrates even at a lower washing and/or drying temperature in the automatic dishwasher. This is also true when using modern dishwashers, which already produce a high drying performance on the part of the machine, as the experiment with the machine c) shows.

Using the formulation 1 and also the reference rinse aid, parts of kitchen- or tableware made of glass, porcelain, metal (=cutlery), Tupperware and styreneacrylonitrile plastic were washed, likewise under the same conditions, in a modified AEG Electrolux (Favorit 60870 model) dishwashers, and the drying was assessed in accordance with EN 50242 and compared. The individual polycyclic test runs were tested here in variable programmes at 50° C. and also at 60° C. in the clear-rinse cycle and without fan.

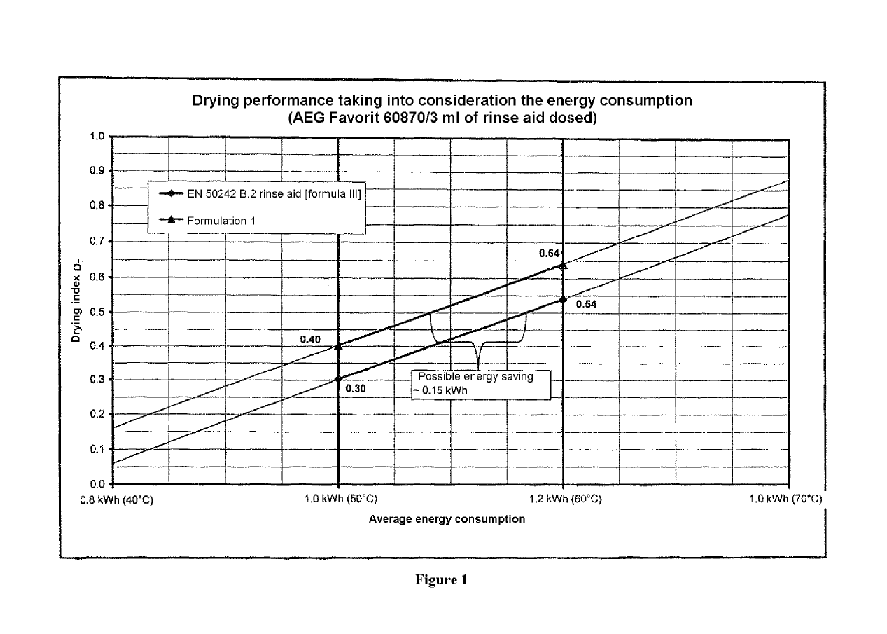

FIG. 1 depicts the results graphically, plotting the drying index T_{D} against the drying temperature. It is found that formulation 1 permits savings, for the same drying performance, compared with the reference rinse aid of around 0.15 kWh per run.

1. A rinse aid comprising
a) at least one surfactant according to the general formula (I) 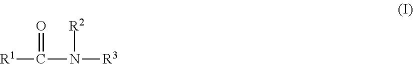
wherein R¹ is a saturated or unsaturated, branched or linear alkyl or alkenyl radical having 6 to 22 carbon atoms, R² is hydrogen or an alkyl radical having 1 to 6 carbon atoms or a radical R³, and R³ is a radical A-(O—CH₂—CHR⁴—)ₘ—OR⁵ and R⁴ and R⁵, independently of one another, are a hydrogen atom or an alkyl radical having 1 to 4 carbon atoms, and A is a group (CH₂)ₙ— or a hydroxyalkyl group having 2 to 6 carbon atoms, and n is an integer from 1 to 6, and m is an integer or fractional number from 1 to 10,
b) at least one hydrotrope,
c) at least one organic hydroxycarboxylic acid, and
d) water.

2. The rinse aid of claim 1, wherein R¹ is a linear saturated alkyl radical having 8 to 18 carbon atoms, R⁴ is hydrogen, n is 2, and m is an integer or fractional number between 1 and 6.

3. The rinse aid of claim 1, wherein the radical R³ is (CH₂)ₙ—(OC₂H₄)ₓ(OC₃H₆)_{y}—OH, n is an integer from 1 to 6, x is a number from 1 to 4, and y is an integer or fractional number from 1 to 4 or has the value zero, wherein the sum of x and y is at most 10.

4. The rinse aid of claim 3, wherein x has a higher value than y, or wherein y has a higher value than x, or wherein x and y are the same.

5. The rinse aid of claim 1, wherein the radical R³ is (CH₂)ₙ—(OC₂H₄)ₓ(OC₃H₆)_{y}—OH, n is an integer from 1 to 6, x is a number from 1 to 4, and y is an integer or fractional numberfrom 1 to 4, wherein the sum of x and y is at most 10.

6. The rinse aid of claim 1, wherein the at least one hydrotrope is selected from the group consisting of cumenesulfonates, xylenesulfonates, alkyl (oligo)glycosides, ethoxylated alkyl (oligo)glycosides, alkylated alkyl (oligo)glycosides, alkylphosphates and/or fatty alcohol alkoxylates.

7. The rinse aid of claim 1, wherein the at least one organic carboxylic acid is selected from the group consisting of mandelic acid, lactic acid, hydroxysuccinic acid, citric acid and tartaric acid.

8. The rinse aid of claim 1, wherein the at least one surfactant is present in an amount from 1 to 80% by weight, based on the weight of the total composition.

9. The rinse aid of claim 1, wherein the at least one hydrotrope is present in an amount from 1 to 30% by weight, based on the weight of the total composition.

10. The rinse aid of claim 1, wherein the at least one organic hydroxycarboxylic acid is present in an amount from 1 to 10% by weight, based on the weight of the total composition.

11. The rinse aid of claim 1, wherein water in present in an amount from 10 to 90% by weight, based on the weight of the total composition.

12. The rinse aid of claim 1, wherein the rinse aid is free from addition products of less than mol of ethylene oxide onto carboxamides having 8 to 20 carbon atoms.

13. The rinse aid of claim 1, wherein the at least one surfactant of the general formula (I) ethylene oxide groups and propylene oxide groups.

14. A method for producing rinse aids or dishwashing detergents comprising: using at least one surfactant of the general formula (I) for producing rinse aids, or for producing dishwashing detergents.

15. A method of using the at least one surfactant of the general formula (I) for improving the drying performance and/or clear rinse performance of rinse aids for dishwashing, or of dishwashing detergents.

16. A method for automatic dishwashing, wherein ware passes through
i) at least one cleaning cycle using a detergent and water,
ii) at least one clear-rinse cycle using a rinse aid and water, and
iii) a final drying cycle,
wherein the temperature in the drying cycle does not exceed 55° C. and wherein the rinse aid of claim 1 is used during the cleaning cycle and/or during the clear-rinse cycle.

17. The method of claim 16, wherein the method for automatic dishwashing achieves a savings in energy in the range of 0.05 to 0.2 kWh compared with a comparable process where a rinse aid without the at least one surfactant of formula (I) is used.

18. The rinse aid of claim 1, wherein the at least one surfactant is present in an amount from 10 to 50% by weight, based on the weight of the total composition.

19. The rinse aid of claim 18, wherein the at least one surfactant is present in an amount from 15 to 40% by weight, based on the weight of the total composition.

20. The rinse aid of claim 1, wherein the at least one surfactant is present in an amount from 15 to 40% by weight, the at least one hydrotrope is present in an amount from 1 to 10% by weight, the at least one hydroxycarboxylic acid is present in an amount from 1 to 5% by weight, and water is present in an amount from 50 to 65% by weight, based on the weight of the total composition.
